(19) United States
(12) Patent Application Publication
(10) Pub. No.: US 2025/0059122 A1
(43) Pub. Date: Feb. 20, 2025

(54) ANTIFOAM / DEFOAM FORMULATION

(71) Applicant: HINDUSTAN PETROLEUM CORPORATION LIMITED, Bengaluru (IN)

(72) Inventors: Sanjoy MUKHERJEE, Bengaluru (IN); Sandip BHOWMIK, Bengaluru (IN); Siva Kesava Raju CHINTHALAPATI, Bengaluru (IN); Shankar JAINENDRAKUMAR, Bengaluru (IN); Sheshachala SRINIVASA NARASIMHA, Bengaluru (IN); Ravi BALASUBRAMANIAM, Andhra Pradesh (IN)

(21) Appl. No.: 18/609,262

(22) Filed: Mar. 19, 2024

(30) Foreign Application Priority Data
Aug. 8, 2023 (IN) 202341053242

(51) Int. Cl. C07C 41/09; C07C 67/08

(52) U.S. Cl. CPC C07C 41/09; C07C 67/08

(57) ABSTRACT

An antifoaming/defoaming compound made of fatty-acid esters of poly-oxyethylene as additives to reduce foaming in aqueous alkaline media and its process of preparation. It is found that C₁₄ fatty acid i.e., myristate esters when used as an antifoaming additive shows exceptional foam reduction tendency ˜90%. To enhance the performance of the additive, there may be provided an antifoaming formulation having an additive and the promoter. The combined anti-foam formulation shows foam reduction of >95%.

### CROSS-REFERENCE TO RELATED APPLICATIONS

The present application claims priority to Indian Patent Application Serial No. 202341053242, filed Aug. 8, 2023, the entirety of which is incorporated herein by reference.

### FIELD OF THE INVENTION

The present invention relates to an antifoaming/defoaming compound having effects of breaking foam and its process of preparation thereof. The present invention also relates to an antifoaming formulation having an additive and the promoter.

### BACKGROUND OF THE INVENTION

According to the classical conceptions, the foams are dispersed systems in which the disperse part is gaseous, but the dispersant is liquid. Numerous industrial processes involving a treatment step of liquid or a treatment step with liquid is often associated with problems of foam formation. These foaming issues are often heightened in the presence of soluble metal salts and insoluble salt particulates, other organic constituents and containments. The foaming of the liquid is contributory of a reduction in the efficiency of the treatment process or the quality of a product.

To mitigate foaming related efficiency losses, additives are used in industrial processes. Additives such as antifoaming/defoaming agents having effects of breaking foam, i.e., a foam breaking effect, or an effect of lowering the foam formation, i.e., a foam controlling effect is used to solve the difficult foaming of liquid. The mechanistic essence of additive based foam control is that the antifoam agent itself accumulates at the air-liquid interface, disrupts the foam's stability, but is unable to form a new, ordered, monomolecular film layer. Commonly used anti-foaming agents are water insoluble oils, such as silicones, certain alcohols, stearates and glycols.

Antifoaming/defoaming additives are often emulsified mixtures of poly-ethers and fatty acids, and/or fatty acid esters. Many additive formulations are fatty acid based emulsion products. Some examples of fatty acid based emulsion products are set forth in the following prior arts.

U.S. Pat. No. 3,705,860A relates to an improved antifoam agents particularly adapted for use in paper and pulp manufacturing processes and prepared from a mixture of non-polar oil, precipitated silica, metallo-organic catalyst, polymethylsilioxane and microcrystalline paraffin wax. These antifoam agents are useful also in latex paint manufacture.

U.S. Pat. No. 3,337,595A relates to new compounds, i.e., fatty acid esters of polyoxypropylated glycerol. These compounds have proven useful for controlling, suppressing and/or preventing foaming of aqueous systems having foaming tendencies.

DE3013292 relates to a process for preparing anti-foams in a free-flowing form which, because of their low melting or softening point, tend to cake when stored.

U.S. Pat. No. 4,451,390A relates to a composition for the control of unwanted foaming comprising Soya bean oil, a mineral oil, a finely divided silica, an unsubstituted fatty acid monoester of glycerol, an unsubstituted fatty acid ester of a polyoxyalkylated sorbitan. These compositions are valuable for the control of foam especially in fermentations since they are stable to sterilization. Also, they may be used in paints since they do not give the defects often associated with silicone antifoams.

Antifoaming/defoaming additives are often emulsified mixtures of poly-ethers and fatty acids, and/or fatty acid esters. Many additive formulations are fatty acid-based emulsion products. Linear organic esters containing two chemically dissimilar hydrophilic and hydrophobic chain-ends are favorable as they can be tailored to be chemically robust yet non-corrosive in nature.

### OBJECTIVE OF THE INVENTION

One objective of the present invention is to provide a fatty-acid esters of poly-oxyethylene as an additive/promoter to reduce foaming in aqueous alkaline media.

Another objective of the present invention is to provide an antifoaming formulation made of fatty-acid esters of poly-oxyethylene as an additive and a tetra-arm derivative as promoter to enhance the foam reducing tendency of the additive.

Another objective of the present invention is to provide an antifoaming formulation that doesn't suffer from phase-separation, easing handling and usage restrictions.

### BRIEF SUMMARY OF THE INVENTION

In the present invention a series of fatty-acid esters of poly-oxyethylene is provided by gradually altering the fatty-acid chain length in a series of fatty acids polyol esters. The selected fatty-acids were further taken for preparing alternative tri-block (A-B-A) and penta-block (A-B-C-B-A) chains to find their relative efficacy. Finally, a tetra-arm [A′-B′]₄C′ type molecule are prepared, which can further enhance disruption of foaming tendencies due to its non-linear architecture. All the designs could be achieved using scalable esterification reactions. Due to chemical tethering of the fatty-acid onto the polyol chains, the resultant liquid materials do not suffer from phase-separation, easing handling and usage restrictions.

In a first aspect of the present disclosure, there is provided an antifoaming promoter having structural formula (I) or (Ia): Linear A-B-C-B-A (I); or Tetra-arm [A′-B′]₄C′ (Ia) wherein: A is moiety from fatty acid, B is moiety from di-alcohol, C is moiety from fatty di-acid, A′ is moiety from fatty alcohol, B′ is moiety from fatty di-acid, and C′ is moiety from tetra alcohol.

In another embodiment, the present invention provides antifoaming promoter, wherein the fatty acid is selected from myristic acid, stearic acid and hexadecenoic acid; the di-alcohol is selected from PEG-400 and polypropylene glycol; the fatty di-acid is selected from sebacic acid, adipic acid and suberic acid.

The antifoaming promoter formula (I), wherein the formula (I) is compound 9:

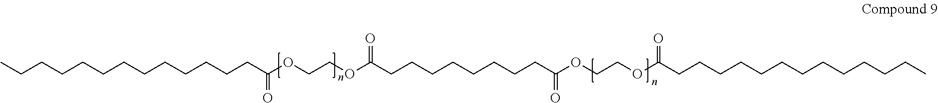

In another embodiment, the present invention provides a method of synthesizing the compound 9 by a process comprising steps of: (a) adding of compound 8 and myristic acid in toluene to obtain a reaction mixture;

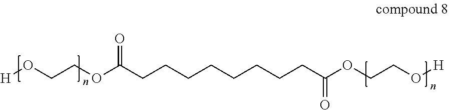

(b) heating the reaction mixture of step (a) with constant stirring for 4 to 8 hours to obtain compound 9.

In one of the preferred features of the present invention the heating of reaction mixture in synthesis of compound 9 is carried out at a temperature of 110° C.

In another embodiment, the present invention provides a method of synthesizing the compound 8 by a process comprising steps of: (a) adding sebacic acid and PEG-400 in toluene to obtain a reaction mixture; and (b) heating the reaction mixture with constant stirring for 4 to 8 hours to obtain compound 8.

In one of the preferred features of the present invention, the heating of reaction mixture in synthesis of compound 8 is carried out at a temperature of 110° C.

In another embodiment, the present invention provides antifoaming promoter of Tetra-arm [A′-B′]₄C′ structure, wherein the fatty alcohol is selected from myristyl alcohol, stearyl alcohol and dodecanol; the fatty di-acid is selected from sebacic acid, adipic acid and suberic acid; the tetra alcohol is selected from pentaerythritol, erythritol and threitol.

The antifoaming promoter formula (Ia), wherein the formula (Ia) is compound 10:

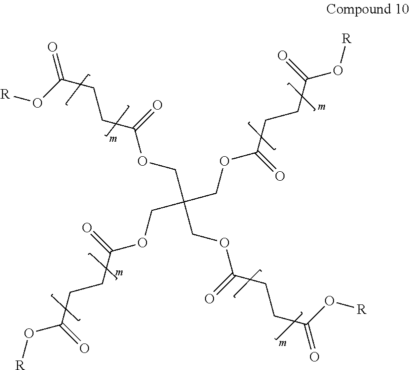

In another embodiment, the present invention provides a method of synthesizing antifoaming compound 10 by a process comprising steps of: (a) adding sebacic acid and myristyl alcohol in toluene to obtain a reaction mixture; (b) heating the reaction mixture of step (a) with constant stirring for 4 to 8 hours; and (c) adding pentaerythritol in the reaction mixture of step (b) and continuing stirring for 8 to 16 to obtain compound 10.

In one of the preferred features of the present invention, the heating of reaction mixture in synthesis of compound 10 is carried out at a temperature of 110° C.

In another embodiment, the present invention provides an antifoaming formulation comprising: (a) an additive, wherein the additive is compound 4

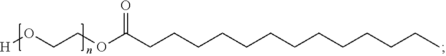

and (b) the promoter as disclosed herein.

In another embodiment, the present invention provides the antifoaming formulation, wherein the promoter is compound 9, compound 10 or combination thereof.

In one of the preferred features of the present invention the antifoaming formulation has ratio of compound 4:compound 10 is 19:1.

In yet another embodiment, the present invention provides a process for foam reduction which comprises incorporating the antifoaming formulation in a material generating the foam, wherein the amount of formulation is from 1 to 20 ppm of the material generating the foam.

In another embodiment, the present invention provides a process for foam reduction, wherein the foam reduction is done in any air liquid interface where liquid phase is alkaline aqueous media.

Further areas of applicability of the present invention will become apparent from the detailed description provided hereinafter. It should be understood that the detailed description and specific examples, while indicating the preferred embodiment of the invention, are intended for purposes of illustration only and are not intended to limit the scope of the invention.

### BRIEF DESCRIPTION OF THE DRAWINGS

The present invention will become more fully understood from the detailed description and the accompanying drawings, wherein:

FIG. 1: illustrates schematic representation of the architectures of synthesized compounds. From left to right: linear di-block A-B type, linear tri-block A-B-A type, linear penta-block A-B-C-B-A type, and tetra-arm [A′-B′]₄C′ type designs.

FIG. 2: illustrates plausible mechanism of foam rupture in presence of additive and promoter.

FIG. 3: illustrates experimental design for calculation of foam reduction efficiency of additive and additive in the presence of promoter in comparison with blank solution.

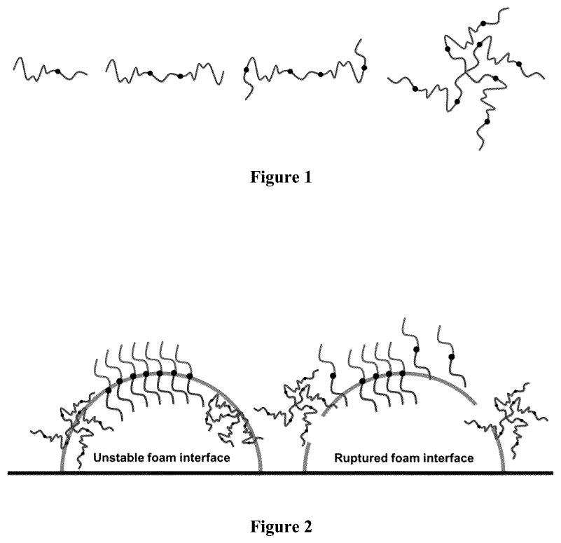

### DETAILED DESCRIPTION OF THE INVENTION

For the purpose of promoting an understanding of the principles of the invention, reference will now be made to the embodiments in the specific language will be used to describe the same. It will nevertheless be understood that no limitation of the scope of the invention is thereby intended, such alterations and further modifications in the illustrated process, and such further applications of the principles of the invention as illustrated therein being contemplated as would normally occur to one skilled in the art to which the invention relates. Unless otherwise defined, all technical and scientific terms used herein have the same meaning as commonly understood by one of ordinary skilled in the art to which this invention belongs. The composition, methods, and examples provided herein are illustrative only and not intended to be limiting.

The articles “a”, “an” and “the” are used to refer to one or to more than one (i.e., to at least one) of the grammatical object of the article.

The terms “comprise” and “comprising” are used in the inclusive, open sense, meaning that additional elements may be included. It is not intended to be construed as “consists of only”.

Throughout this specification, unless the context requires otherwise the word “comprise”, and variations such as “comprises” and “comprising”, will be understood to imply the inclusion of a stated element or step or group of element or steps but not the exclusion of any other element or step or group of element or steps.

Unless defined otherwise, all technical and scientific terms used herein have the same meaning as commonly understood by one of ordinary skill in the art to which this disclosure belongs. Although any methods and materials similar or equivalent to those described herein can be used in the practice or testing of the disclosure, the preferred methods and materials are now described. All publications mentioned herein are incorporated herein by reference.

In the present invention linear di-block esters of a poly-ether derivative and a fatty-acid derivative were synthesized using 1:1 molar ratio of the constituents, refluxed in toluene as a solvent at 110° C.

Linear tri-block esters where the central block is a poly-ether derivative were synthesized using 1:2 molar ratios of the polyether and fatty-acid as constituents, refluxed in toluene as a solvent at 110° C.

Linear tri-block esters where the central block ester is a linear di-acid was prepared using 1:2 molar ratios of the di-acid and polyether, refluxed in toluene at 110° C.

The same could be used for further esterification of the poly-ether open chain ends, to prepare linear penta-block esters. Di-block linear esters of di-acids and fatty-alcohols, when coupled with pentaerythritol under similar reaction conditions, yielded the tetra-arm derivatives (e.g., compound 10).

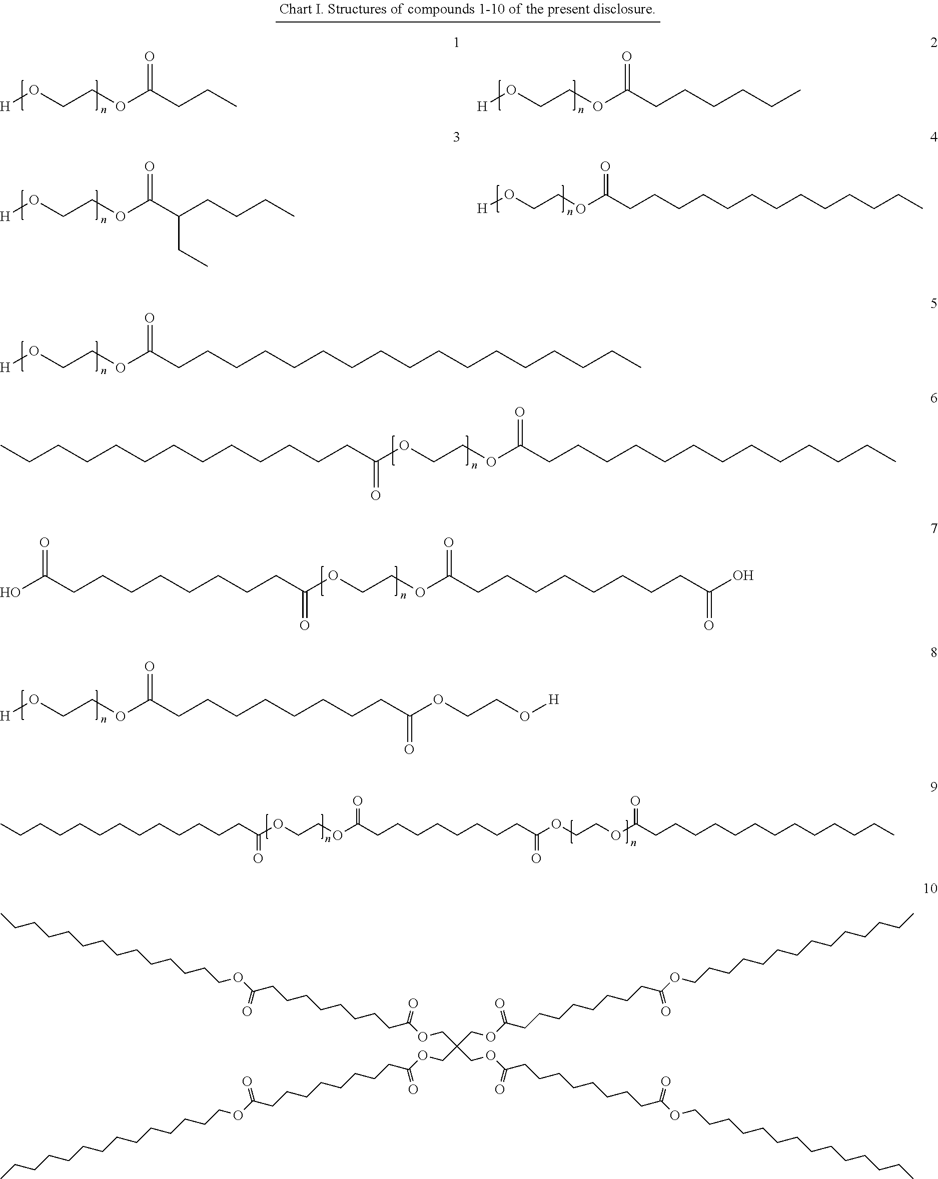

TABLE 1
Structural Formula of synthesized compounds 1-10

| Structure-Formula | Polyol | Alkyl-acid component | Name |
|---|---|---|---|
| Linear A-B | PEG-400 (A) | Butanoic acid (B) | 1 |
| | PEG-400 (A) | Heptanoic acid (B) | 2 |
| | PEG-400 (A) | 2-ethyl hexanoic acid (B) | 3 |
| | PEG-400 (A) | Myristic acid (B) | 4 |
| | PEG-400 (A) | Stearic acid (B) | 5 |
| Linear A-B-A | PEG-400 (B) | Myristic acid (A) | 6 |
| | PEG-400 (B) | Sebacic acid (A) | 7 |
| | PEG-400 (A) | Sebacic acid (B) | 8 |
| Linear A-B-C-B-A | PEG-400 (B) | Sebacic (C), and | 9 |
| | | Myristic acid (A) | |
| Tetra-arm | Pentaerythritol | Sebacic (B′), and | 10 |
| [A′-B′]₄C′ | (C′) | Myristyl alcohol (A′) | |

### EXAMPLES

The present disclosure with reference to the accompanying examples describes the present invention. However, those of skill in the art should, in light of the present disclosure, appreciate that many changes can be made in the specific embodiments which are disclosed and still obtain a like or similar result without departing from the spirit and scope of the invention. It is understood that the examples are provided for the purpose of illustrating the invention only and are not intended to limit the scope of the invention in any way.

### Example 1: Synthesis of Compounds 1-5

40 g of PEG-400 (100 mmol) and 8.81 g of butyric acid (100 mmol) were taken with 100 mL toluene in a 250 mL round-bottom flask. The reaction mixture was heated to 110° C. under constant stirring for 8 hours and the released water was separated from the reflux media using a dean-stark trap. After completion of the reaction, solvent was evaporated under vacuum, and the desired product (1) was obtained a colorless liquid (yield >99%). Similar procedure was followed for compounds 2-5 where the 100 mmol of the corresponding fatty-acid was taken instead of butyric-acid. All compounds were obtained as colorless liquids (Table 1).

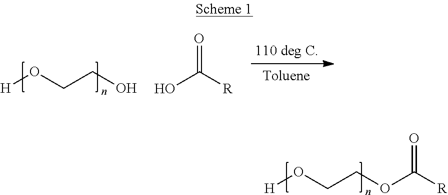

where R is any linear or branched alkyl group, and n ranges from 10-25.

### Example 2: Synthesis of Compounds 6 and 7

40 g of PEG-400 (100 mmol) and 45.70 g of myristic acid (200 mmol) were taken with 100 mL toluene in a 250 mL round-bottom flask. The reaction mixture was heated to 110° C. under constant stirring for 8 hours and the released water was separated from the reflux media using a dean-stark trap. After completion of the reaction, solvent was evaporated under vacuum, and the desired product (6) was obtained a colorless liquid (yield >99%). Similar procedure was followed for compound 7 where the 200 mmol of the corresponding di-acid was taken instead of myristic acid. Compound 7 was isolated as white colored solid (Table 1).

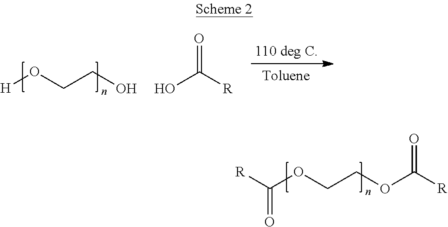

where R is any linear alkyl group, and n ranges from 10-25.

### Example 3: Synthesis of Compound 8

20.22 g of sebacic acid (100 mmol) and 80 g of PEG-400 (200 mmol) were taken with 100 mL toluene in a 250 mL round-bottom flask. The reaction mixture was heated to 110° C. under constant stirring for 8 hours and the released water was separated from the reflux media using a dean-stark trap. After completion of the reaction, solvent was evaporated under vacuum, and the desired product (8) was obtained as colorless liquid (yield >98%) (Table 1).

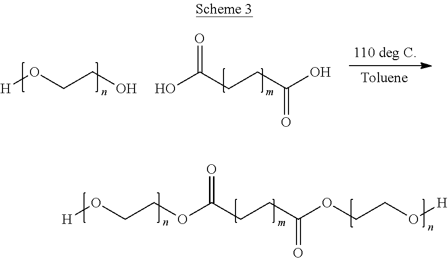

where n ranges from 10-25, and m is 8.

### Example 4: Synthesis of Compound 9

96.6 g of compound 8 (100 mmol) and 45.70 g of myristic acid (200 mmol) were taken with 100 mL toluene in a 250 mL round-bottom flask. The reaction mixture was heated to 110° C. under constant stirring for 8 hours and the released water was separated from the reflux media using a dean-stark trap. After completion of the reaction, solvent was evaporated under vacuum, and the desired product (9) was obtained as colorless liquid (yield >97%) (Table 1).

¹H NMR (500 MHz, CDCl₃): σ 4.2 (m, 8H), 3.7 (m, 40H), 2.4 (m, 8H), 1.7 (m, 8H), 1.3 (m, 48H), 0.9 (t, 6H). FTIR 2920 cm⁻¹, 2850 cm⁻¹, 1735 cm⁻¹, 1450 cm⁻¹, 1350 cm⁻¹, 1250 cm⁻¹, 1100 cm⁻¹, 725 cm⁻¹, 695 cm⁻¹.

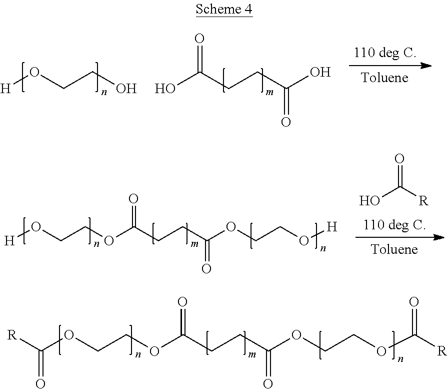

where n ranges from 10-25, and m is 8.

### Example 5: Synthesis of Compound 10

20.22 g of sebacic acid (100 mmol) and 21.4 g of myristyl alcohol (100 mmol) were taken with 100 mL toluene in a 250 mL round-bottom flask. The reaction mixture was heated to 110° C. under constant stirring for 8 hours and the released water was separated from the reflux media using a dean-stark trap. Further, 3.4 g of pentaerythritol (25 mmol) was added in the reaction mixture and the reaction was continued for another 16 hrs. After completion of the reaction, the solvent was evaporated, and the desired compound was obtained as a low melting white-colored solid (Table 1).

¹H NMR (500 MHz, CDCl₃): σ 4.25 (t, 8H), 3.7 (m, 8H), 2.3 (m, 16H), 1.6 (m, 24H), 1.3 (m, 120H), 0.9 (t, 12H). FTIR 2950 cm⁻¹, 2915 cm⁻¹, 2850 cm⁻¹, 1731 cm⁻¹, 1700 cm⁻¹, 1420 cm⁻¹, 1300 cm⁻¹, 1215 cm⁻¹, 1175 cm⁻¹, 715 cm⁻¹.

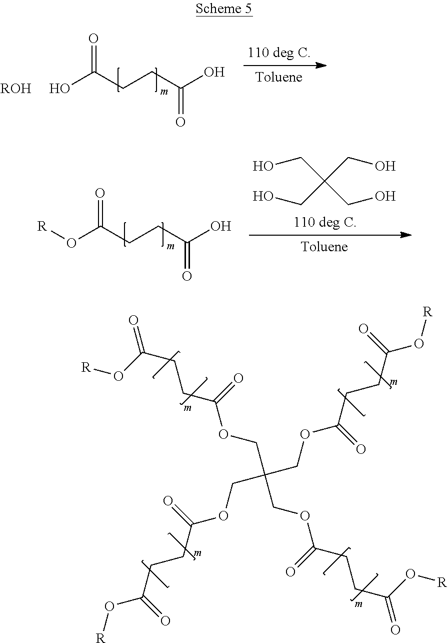

### Example 6: Evaluation of the Formulations

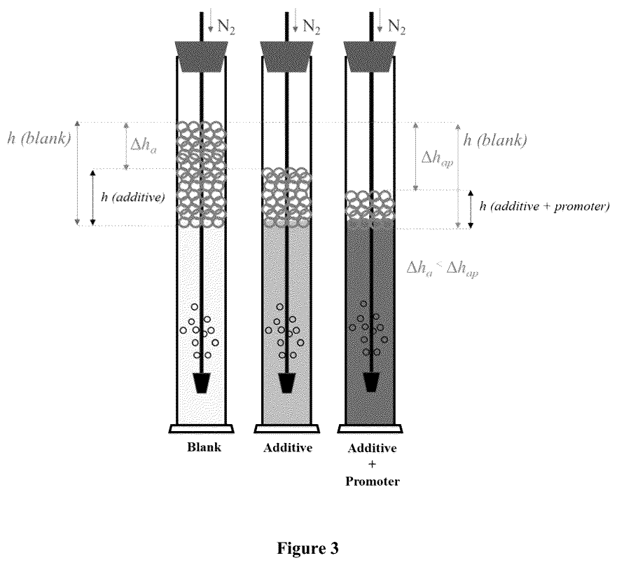

The foaming tests were carried out by measuring equilibrium foam heights. For example, a blank/reference and an additive containing test solutions were exposed to a constant purge of N₂ (nitrogen) and the equilibrium heights were compared to determine the percentage of foam reduction as an effect of the additive. A solution of 35 weight % MDEA (methyl di-ethanol amine) with 0.5 weight % Fe(NO₃)₃·9H₂O (iron nitrate nonahydrate) was taken as the reference/blank solution (FIG. 3 & Table 2).

Equal volume of a blank solution/reference solution is compared with an additive containing solution under equilibrium nitrogen (N₂) purging i.e., all the conditions in two parallel set-ups are maintained same except that one of the solutions contain the additive formulation.

The foam-reduction is calculated as follows: % Foam reduction=Δh÷h (blank)×100.

TABLE 2
The % Foam reduction obtained for the additives and promoter

| Additive composition | | | % Foam |
|---|---|---|---|
| Additive | Promoter | Dosage | Reduction |
| Compound 1 (100%) | — | 5 ppm | Negligible |
| Compound 2 (100%) | — | 5 ppm | Negligible |
| Compound 3 (100%) | — | 5 ppm | ~60% |
| Compound 4 (100%) | — | 5 ppm | ~90% |
| Compound 4 (100%) | — | 1 ppm | ~85% |
| Compound 5 (100%) | — | 5 ppm | ~70% |
| Compound 4 (95%) | Compound 6 (5%) | 5 ppm | ~90% |
| Compound 4 (95%) | Compound 7 (5%) | 5 ppm | ~90% |
| Compound 4 (95%) | Compound 8 (5%) | 5 ppm | ~90% |
| Compound 4 (95%) | Compound 9 (5%) | 5 ppm | >90% |
| Compound 4 (95%) | Compound 10 (5%) | 5 ppm | >95% |
| Compound 4 (95%) | Compound 10 (5%) | 1 ppm | >90% |

Comprehensive screening of the different additives helped to identify the best additive (i.e., Compound 4) which is further taken up with different promoters to find the best possible formulation (i.e., Compound 4 and Compound 10 in 19:1 ratio). The tetra-arm designs act as performance enhancers for linear systems as the non-linear structure would destabilize the foam surfaces to greater effects. The finalized formulations could reduce the foaming of the media up to 95% extent when compared to reference solutions.

While the invention has been described with respect to specific examples including presently preferred modes of carrying out the invention, those skilled in the art will appreciate that there are numerous variations and permutations of the above described systems and techniques. It is to be understood that other embodiments may be utilized and structural and functional modifications may be made without departing from the scope of the present invention. Thus, the spirit and scope of the invention should be construed broadly as set forth in the appended claims.

What is claimed is:

1. An antifoaming promoter having structural formula (I) or (Ia):
Linear A-B-C-B-A (I); or
Tetra-arm [A′-B′]₄C′ (Ia)
wherein:
A is moiety from fatty acid,
B is moiety from di-alcohol
C is moiety from fatty di-acid.
A′ is moiety from fatty alcohol,
B′ is moiety from fatty di-acid, and
C′ is moiety from tetra alcohol.

2. The antifoaming promoter according to claim 1, wherein the fatty acid is selected from myristic acid, stearic acid and hexadecenoic acid, the di-alcohol is selected from PEG-400 and polypropylene glycol, the fatty di-acid is selected from sebacic acid, adipic acid and suberic acid.

3. The antifoaming promoter according to claim 1, wherein the formula (I) is compound 9:

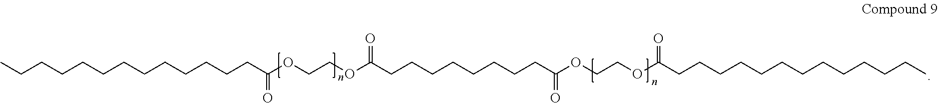

4. A method for preparing antifoaming compound 9 according to claim 3, comprising steps of:
(a) adding of compound 8 and myristic acid in toluene to obtain a reaction mixture; 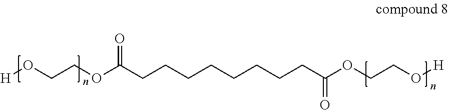
(b) heating the reaction mixture of step (a) with constant stirring for 4 to 8 hours to obtain compound 9.

5. The method according to claim 4, wherein the heating of a reaction mixture is carried out at a temperature of 110° C.

6. The method according to claim 4, wherein the compound 8 is synthesized by a process comprising steps of:
(a) adding sebacic acid and PEG-400 in toluene to obtain a reaction mixture; and
(b) heating the reaction mixture with constant stirring for 4 to 8 hours to obtain compound 8.

7. The method according to claim 6, wherein the heating of a reaction mixture is carried out at a temperature of 110° C.

8. The antifoaming promoter according to claim 1, wherein the fatty alcohol is selected from myristyl alcohol, stearyl alcohol and dodecanol; the fatty di-acid is selected from sebacic acid, adipic acid and suberic acid; the tetra alcohol is selected from pentaerythritol, erythritol and threitol.

9. The antifoaming promoter according to claim 1, wherein the formula (Ia) is compound 10:

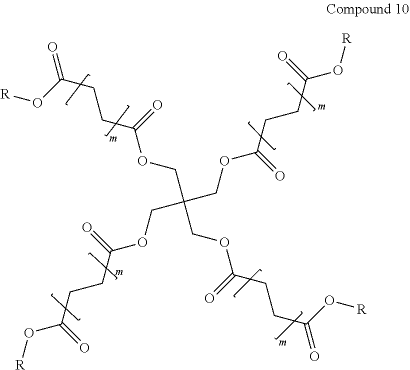

10. A method for preparing antifoaming compound 10 according to claim 9, comprising steps of:
(a) adding sebacic acid and myristyl alcohol in toluene to obtain a reaction mixture;
(b) heating the reaction mixture of step (a) with constant stirring for 4 to 8 hours; and
(c) adding pentaerythritol in the reaction mixture of step (b) and continuing stirring for 8 to 16 to obtain compound 10.

11. The method according to claim 10, wherein the heating of a reaction mixture is carried out at a temperature of 110° C.

12. An antifoaming formulation comprising:
(a) an additive, wherein the additive is compound 4 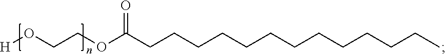
and
(b) the promoter as defined in claim 1.

13. The antifoaming formulation according to claim 12, wherein the promoter is compound 9, compound 10 or combination thereof.

14. The antifoaming composition formulation according to claim 13, wherein the ratio of compound 4 to compound 10 is 19:1.

15. A process for foam reduction which comprises incorporating the formulation according to claim 12 in a material generating the foam, wherein the amount of formulation is from 1 to 20 ppm of the material generating the foam.
